(19) United States
(12) Patent Application Publication
(10) Pub. No.: US 2020/0392709 A1
(43) Pub. Date: Dec. 17, 2020

(54) Toilet with Health and Wellness Sensors in the Lid

(71) Applicant: Medic, Inc., Provo, UT (US)

(72) Inventors: David R. Hall, Provo, UT (US); David Crismon, Herriman, UT (US); Joshua Larsen, Spanish Fork, UT (US); Jared Reynolds, Spanish Fork, UT (US)

(73) Assignee: Medic, Inc., Provo, UT (US)

(21) Appl. No.: 16/904,010

(22) Filed: Jun. 17, 2020

Related U.S. Application Data
(63) Continuation-in-part of application No. 16/828,210, filed on Mar. 24, 2020.
Application No. 16/828,210 is a continuation-in-part of application No. 16/709,028, filed on Dec. 10, 2019.
Application No. 16/828,210 is a continuation-in-part of application No. 16/709,163, filed on Dec. 10, 2019.
Application No. 16/828,210 is a continuation-in-part of application No. 16/709,505, filed on Dec. 10, 2019.
Application No. 16/828,210 is a continuation-in-part of application No. 16/709,549, filed on Dec. 10, 2019.
(60) Provisional application No. 62/862,564, filed on Jun. 17, 2019.
(60) Provisional application No. 62/888,683, filed on Aug. 19, 2019.
(60) Provisional application No. 62/907,408, filed on Oct. 9, 2019.
(60) Provisional application No. 62/907,432, filed on Sep. 27, 2019.
(60) Provisional application No. 62/862,597, filed on Jun. 17, 2019.
(60) Provisional application No. 62/862,574, filed on Jun. 17, 2019.
(60) Provisional application No. 62/862,577, filed on Jun. 17, 2019.
(60) Provisional application No. 62/888,663, filed on Aug. 19, 2019.
(60) Provisional application No. 62/892,927, filed on Aug. 28, 2019.

(51) Int. Cl. E03D 9/00; A47K 13/24; A61B 10/00; A61B 5/0205; A61B 5/00; G01N 1/22

(52) U.S. Cl. CPC E03D 9/00; A47K 13/24; A61B 10/00; G01N 33/493; A61B 5/6891; G01N 1/2226; A61B 5/02055

(57) ABSTRACT

An analytical toilet comprising a bowl for receiving excreta from a user; a lid adapted to cover the bowl; and a first health and wellness sensor in the lid for measuring a characteristic of the user's excreta is disclosed.

### CROSS-REFERENCE TO RELATED APPLICATIONS

This application is a Continuation-in-Part of U.S. patent application Ser. No. 16/828,210 titled “Toilet with Lid for Sealing Bowl for Analysis and Washing” filed on 24 Mar. 2020, which is a Continuation-in-Part of U.S. patent application Ser. No. 16/709,028 titled “Toilet with Internal Frame and Shroud” filed 10 Dec. 2019 which claims priority to 62/862,564 titled “Toilet with Frame, Bowl and Shroud” filed on 17 Jun. 2019; Ser. No. 16/709,163 titled “Toilet with Internal Lifting System” filed 10 Dec. 2019 which claims priority to 62/888,683 titled “Toilet with Multiple Point Lifting System” filed 19 Aug. 2019; Ser. No. 16/709,505 titled “Toilet with Personalized Lifting System” filed 10 Dec. 2019 which claims priority to 62/907,408 titled “Toilet with Personalized Lifting System for Bowl and Seat” filed on 9 Oct. 2019; and Ser. No. 16/709,549 titled “Toilet with User Weight-Driven Seat Lift Profile” filed 10 Dec. 2019 which claims priority to 62/907,432 titled “Toilet with User Weight Driven Seat Lift Profile” filed on 27 Sep. 2019; each of which are incorporated herein by reference in their entireties. This application also claims priority to U.S. Provisional Patent Application No. 62/862,597 titled “Toilet Lid with Sensors for Analysis of Excreta and Providing Health Information” filed 17 Jun. 2019; 62/862,574 titled “Toilet with Imaging Analysis of Fecal Matter” filed on 17 Jun. 2019; 62/862,577 titled “Toilet Lid for Sealing Bowl During Analysis and Washing” filed on 17 Jun. 2019; 62/888,663 titled “Toilet with Suspended Bowl to Measure Excreta Mass” filed 19 Aug. 2019; and 62/892,927 titled “Toilet with Visual Indicator of Health and Wellness Information” filed 28 Aug. 2019; each of which are incorporated herein by reference in their entireties.

### TECHNICAL FIELD

The present disclosure relates to analytical toilets. More particularly, it relates to analytical toilets that seal the bowl to perform analytical tests and for cleaning.

### BACKGROUND

The ability to track an individual's health and wellness is currently limited to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

At present, there is no easy, hands-off way of measuring human waste weight, volume, density, content, or other characteristics. While each can be done, there is no know automated method integrated in a toilet. Weighing an entire toilet is difficult and traditional toilets do not hold the entire urine event in the bowl, so the best that can be done is to measure the weight of a person before and after using the toilet. This could measure total excreta mass, but there would be no distinction between liquid and solid waste. Also, when you are measuring a small change in weight from a much larger weight (like the weight of waste compared to the weight of an adult) the measurement will typically be less accurate.

Just a few examples of smart toilets and other bathroom devices can be seen in the following U.S. Patents and Published Applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; US 2018/0165417, entitled “Bathroom Telemedicine Station”; U.S. Ser. No. 15/222,267, entitled “THIN WEIGHT SCALE.” The disclosures of all these patents and applications are incorporated by reference in their entireties.

### SUMMARY

In a first aspect, the disclosure provides analytical toilet comprising a bowl for receiving excreta from a user; a lid adapted to cover the bowl; and a first health and wellness sensor in the lid for measuring a characteristic of the user's excreta.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a perspective view of a first exemplary toilet according to the present disclosure in a closed position.

FIG. 2 is a perspective view of the toilet of FIG. 1 with the lid opened and an exemplary embodiment of handles deployed.

FIG. 3 is a side plan view of a second exemplary toilet according to the present disclosure with the shroud removed and the seat in a lowered position for sitting.

FIG. 4 is a side cross-sectional view of the toilet of FIG. 1.

FIG. 4A is close-up view of the seat as shown in FIG. 4.

FIG. 5 is a perspective view of an exemplary embodiment of a toilet with a lid and seat having health and wellness sensors according to the present disclosure.

FIG. 6 is a side view of a user sitting on the toilet of FIG. 5.

FIG. 7A is a top view of a second exemplary embodiment of a toilet seat with a floating hinge according to the present disclosure.

FIG. 7B is a side view of the toilet seat of FIG. 7A.

FIG. 7C is a cross-sectional view of the hinge of the toilet seat of FIG. 7A taken within Line C.

FIG. 7D is a cross-sectional view of the hinge of the toilet seat of FIG. 7A taken within Line D in FIG. 7B.

FIG. 8A is a first exploded perspective view of the hinge of the toilet seat of FIG. 7A.

FIG. 8B is a second exploded perspective view of the hinge of the toilet seat of FIG. 7A.

FIG. 9A is a side view of the internal parts of the hinge of FIG. 7A in a first position.

FIG. 9B is a side view of the internal parts of the hinge of FIG. 7A in a second position.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, the term “excreta” refers to any substance released from the body including urine, feces, menstrual discharge, gases, and anything contained or excreted therewith.

As used herein, the term “toilet” is meant to refer to any device or system for receiving human excreta, including urinals.

As used herein, the term “bowl” is meant to refer to the portion of a toilet that is designed to receive excreta.

As used herein, the term “frame” refers to the portion of the toilet below and around the bowl supporting it, the seat, and any other toilet components.

As used herein, the term “shroud” is meant to refer to the outer surface of the toilet enclosing and covering the frame and typically other toilet components.

As used herein, the term “actuator” is intended to have a relatively broad meaning, referring to any mechanical device for moving the seat and/or bowl. Various types of actuators are suitable for use in the invention. The preferred actuators use hydraulic or pneumatic cylinders. Alternatively, the actuators use screws, levers, hydraulics, pistons, or some other mechanism to raise and lower the seat and/or bowl.

As used herein, the term “processor” is meant to refer to logic circuitry that processes data from the sensors, applies algorithms such as computer vision to identify a user or determine physical characteristics of a user, and instructs a digital controller to adjust the toilet seat and/or bowl position based on the preferences of a known user or characteristics of an unknown (i.e., unidentified) user.

As used herein, the term “position,” as in the position of the bowl, seat or footrest, is intended to refer to the height and/or the angle of the device, with respect to the floor.

As used herein, the term “angle,” as in angle of the seat, is intended to refer to the angle of the seat versus the horizontal floor (i.e., 0° angle) so that increasing the angle refers to the seat approaching perpendicular (i.e., 90° angle).

As used herein, the term “weight” is meant to refer to the mass of a user or their excreta.

As used herein, the term “floating” refers to a component that is supported by weight sensors independently of other components and does not support other components (e.g., a floating bowl does not support the weight of the seat).

As used herein, the term “removable” refers to any portion of the shroud that can be moved to allow access to the interior components of the toilet whether such portion is detached from the toilet or merely moved (e.g., slid over or pivoted on at least one hinges) to provide access.

As used herein, the term “sensor” is meant to refer to any device for detecting and/or measuring a property of a person or of a substance regardless of how that property is detected or measured, including the absence of a target molecule or characteristic.

As used herein, the term “imaging sensor” is meant to refer to any device for detecting and/or measuring a property of a person or of a substance that relies on electromagnetic radiation of any wavelength (e.g., visible light, infrared light, xray) or sound waves (e.g., ultrasound) to view the surface or interior of a user or substance. The term “imaging sensor” does not require that an image or picture is created or stored even if the sensor is capable of creating an image.

As used herein, the terms “weight sensor” and “load cell” are intended to have a relatively broad meaning, referring to a transducer, specifically a force transducer that converts a force such as tension, compression, pressure, or torque into an electrical signal that can be measured and standardized. As the force applied to the weight sensor or load cell increases, the electrical signal changes proportionally.

As used herein, the term “transparent” is meant to refer to materials that allow visible light and/or non-visible light (e.g., ultraviolet, infrared, etc.) to pass through the material. A material need not be transparent to all wavelengths to be considered transparent.

### Exemplary Embodiments

The present disclosure relates to a toilet with a bowl and seat supported by a frame. The frame includes at least one lifting mechanism adapted to raise and lower the seat and/or bowl. In some embodiments, there may be separate frames for supporting and adjusting the seat and for supporting and adjusting the bowl. Preferably, the lifting mechanism is further adapted to change the angle of the seat.

In a preferred embodiment, the toilet comprises a motorized seat lift that is integral with and internal to the toilet. It also provides customized lifting motions set to individual user's preferences. The disclosed toilet looks and feels like a conventional toilet and can help prevent fall by keeping a person more supported during a lift and help them to their feet at a height appropriate for the individual.

In a preferred embodiment, the adjustable frame allows the toilet to be both shorter and taller than conventional toilets. This makes the toilet more comfortable and easier to use for both tall and short users, including children, without the need for special stools or spacers.

In various exemplary embodiments, the bowl is supported by a frame including at least one actuator. In various exemplary embodiments, the seat is supported a frame comprising at least one actuator. In various exemplary embodiments, the frame comprises two, three, four, or more actuators for the bowl and/or seat. In a more preferred embodiment, the frame comprises three pairs of actuators to move the seat. Preferably, the actuators are controlled by a digital controller that coordinates the actions of the actuators to thereby provide a smooth lifting and lower at the desired speed. More preferably, the controller is programmable, so that users can program the lifting and lowering of the seat to their preference. Even more preferably, the toilet includes a user identification module, so that the user is automatically identified as he or she approaches or sits on the toilet.

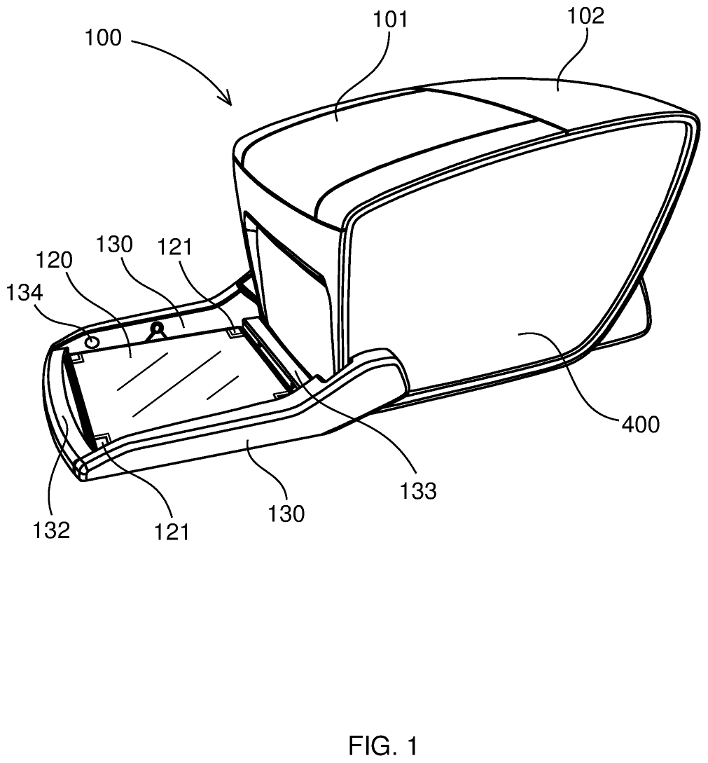

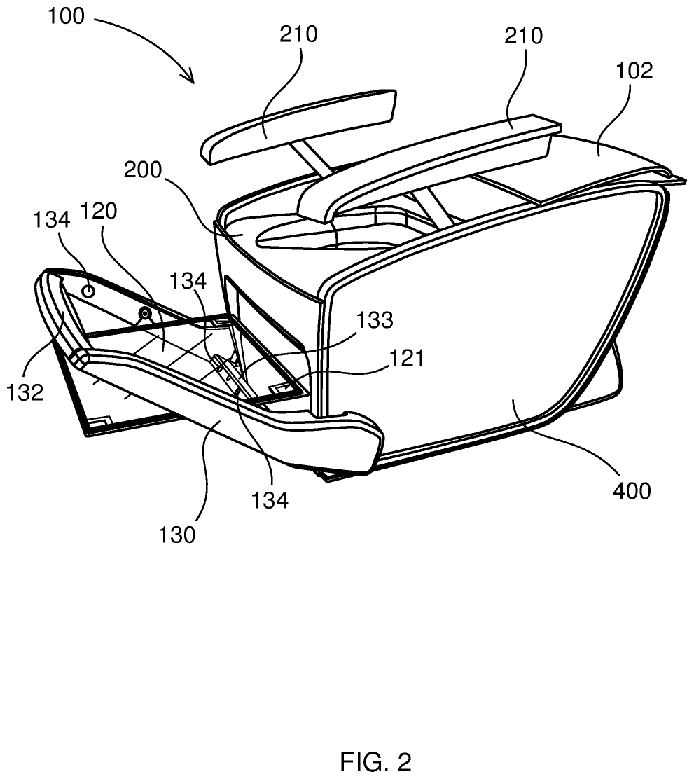

Referring to FIG. 1, an exemplary embodiment of a toilet is shown with a closed lid 101, lowered foot platform 120, and retracted handles 210. FIG. 2 shows the toilet with an open lid 101, raised foot platform 120, and extended handles 210. In a preferred embodiment, handles 210 are adapted to be readily ripped by a user, especially while sitting or rising. In a preferred embodiment, the handles 210 move while the seat 200 is lowering and lifting a user to enable them to grip the handles 210 and use them for support during sitting and rising. The toilet is enclosed with a shroud 400.

In various exemplary embodiments, the shroud 400 covers all the internal support components that comprise the frame as well as the bowl 300 (i.e., the bowl is covered except for the top that would be covered by a lid). In preferred embodiments, the shroud 400 comprises at least one sections of articulated or flexible (e.g., elastic, material) assembled such that the shroud 400 can contract or expand as the frame 110 moves the bowl 300 and/or seat 200. Other sections of the shroud may be rigid. In various exemplary embodiments, the at least some portions of the shroud are removable or openable to allow access to the interior of the toilet frame. The interior of the toilet frame may include, among other things, plumbing connections, fluid supply lines, support structures, health and wellness assessment devices, electronic circuits, digital devices (e.g., processors, memory), storage tanks, and communication modules. In various preferred embodiments, cover 102 opens to allow access to the interior of the frame.

In various exemplary embodiments, the shrouds 400 are preferably designed for aesthetic and other purposes. For example, the shrouds 400 can be made in various colors to suit the decor of the bathroom the toilet is installed in. Besides colors, the shrouds 400 can include patterns or graphics so that the toilet 100 “makes a statement” in the bathroom.

In various exemplary embodiments, the handles 210 include at least one buttons, switches, sensors, etc. through which the user may control the seat, including, but not limited to, starting and stopping the seat 200, reversing seat movement direction, adjusting the position or angle of the seat 200, and adjusting the height of the bowl 300, seat 200, or foot platform 120.

In various exemplary embodiments, the handles 210 may include at least one health and wellness sensors. The sensors may be used to measure how hard the user is gripping and provide feedback to the lift control. For example, if a user suddenly increases their grip it is likely that they are struggling with standing or with their balance. Grip pressure may also be tracked over time. The sensors may also include detection for heart rate, body temperature, hand health indicators, light sensors, electrocardiogram, pulse, blood test (e.g., using a finger prick), etc. Toilet handles with health and wellness sensors are disclosed in U.S. Patent Pub. No. 2018/0084959, the entire disclosure of which is incorporated herein by reference in its entirety.

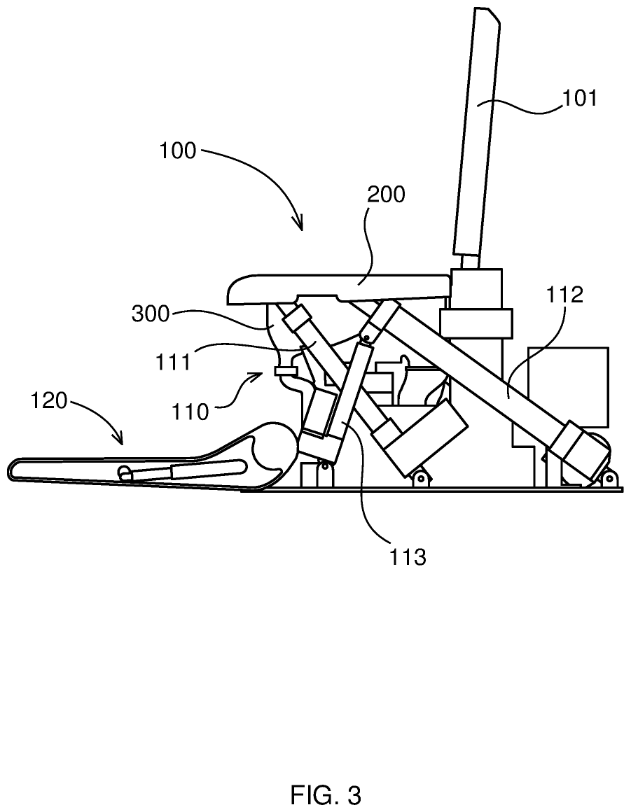

Referring to FIG. 3, an exemplary embodiment of a toilet 100 is shown. The toilet 100 includes a frame 110, bowl 300 supported by the frame 110, a lid 101, and a seat 200 also supported by the frame 110. In a preferred embodiment, the seat 200 is supported by two sets of seat actuators 111, 112, and 113, which can raise and lower the seat and change its angle to tile forward or back. In other embodiments, the number and arrangement of actuators may vary.

In a preferred embodiment, a pair of first seat actuators 111 are pivotally connected to a front area of the seat 200 and a pair of second seat actuators 112 are pivotally connected to a back area of the seat 200 providing support at four “corners” of the seat 200. The four seat actuators 111 and 112 are also pivotally attached to the frame 110. An additional pair of third seat actuators 113 are pivotally attached to the frame 110 at one end and pivotally attached to the second pair of seat actuators 112 at their other end. In another embodiment, the third seat actuators 113 are attached to the first pair of seat actuators 111.

FIG. 3 shows the toilet 100 with the seat 200 fully lowered to just above the top of the bowl 300. Actuators 111, 112, and 113 allow the seat to be adjusted for a variety of users from small children to tall adults.

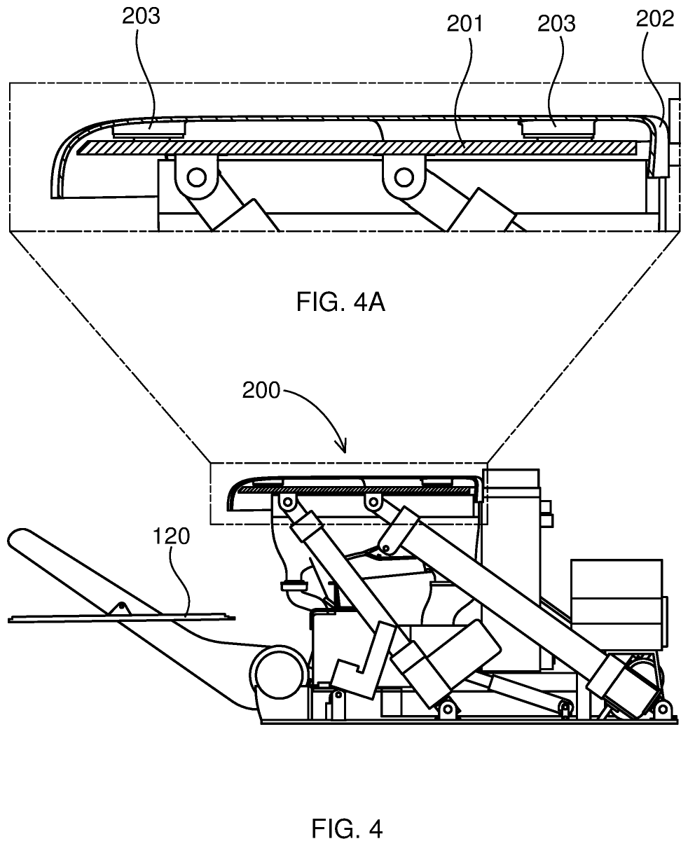

Referring to FIGS. 4 and 4A, an exemplary embodiment of a floating toilet seat 200 according to the present disclosure is shown. The seat includes a rigid seat support 201 covered by a shroud 202. Weight sensors 203 are placed between the seat support 201 and floating cover 202. In various other embodiments, the weight sensors may be placed in a variety of places such as under the actuators or under the frame.

In various exemplary embodiments, the toilet continuously measures weight load on both the seat 200 and the foot platform 120. In a preferred embodiment, the system weighs the user on the foot platform 120 prior to the user placing any weight against the seat 200 and records that as the user's total weight. In some embodiments, the system calculates a user total weight using both the seat weight sensors 203 and foot platform weight sensors 121 after the user seated with the seat 200 fully lowered. In various exemplary embodiments, the system calculates the percentage of user body weight on the foot platform 120. The angle of the seat 200 is limited from raising based on the percentage of weight on the foot platform 120. This prevents the seat 200 from tipping to an angle that will not support the user's weight prior to the user being on their feet. The platform 120 is preferably comprised of a transparent material to facilitate the use of imaging sensors under the platform 120 for analyzing the bottom of the user's feet.

In various exemplary embodiments, the urine is tested for a variety of properties including volume, flow rate, color, weight, density, content, temperature, clarity, pH, settled gradient, and flow geometry. This may be done with a variety of sensors or testing methods including MOS, CCD, spectrometers, volume measurement devices, weight sensors, temperature gauges, chromatographs, mass spectrometers, and gas analyzers.

Referring to FIG. 5, an exemplary embodiment of an analytical toilet 1100 is shown. In various exemplary embodiments, sensors in the lid collect data with the lid 1101 closed. Such first sensors 1102A may include imaging sensors such as CCD, MOS/CMOS, IR camera, visible light camera, 3D camera, near-IR camera, and UV camera. They may also include spectrometers that measure light that has passed through the excreta or reflected off of it. The interior of the bowl 1300 may be illuminated with visual, infrared, near infrared, ultraviolet, or other wavelengths by light sources 1103 to enable use of these sensors.

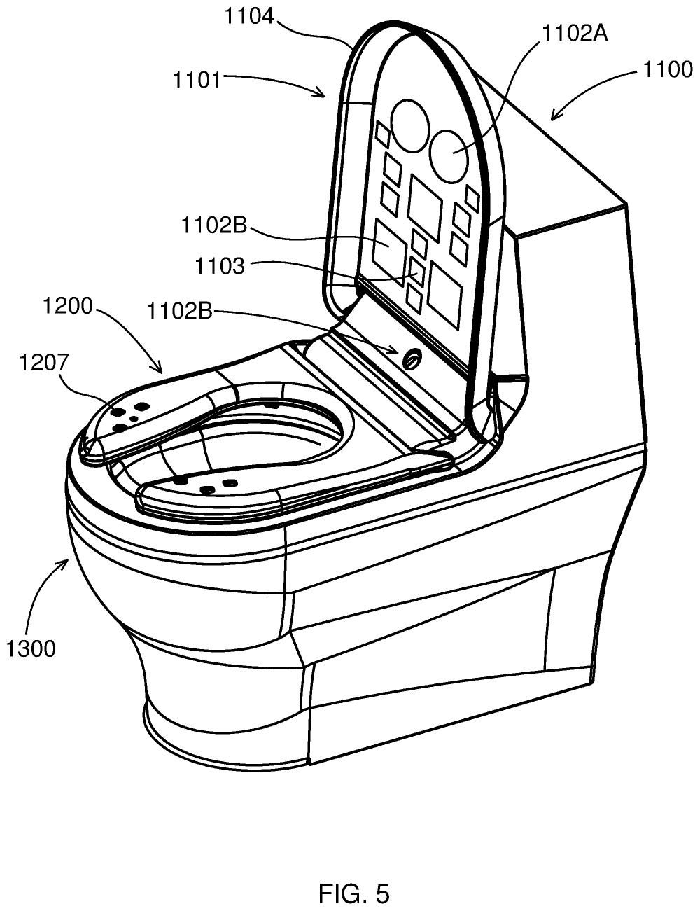

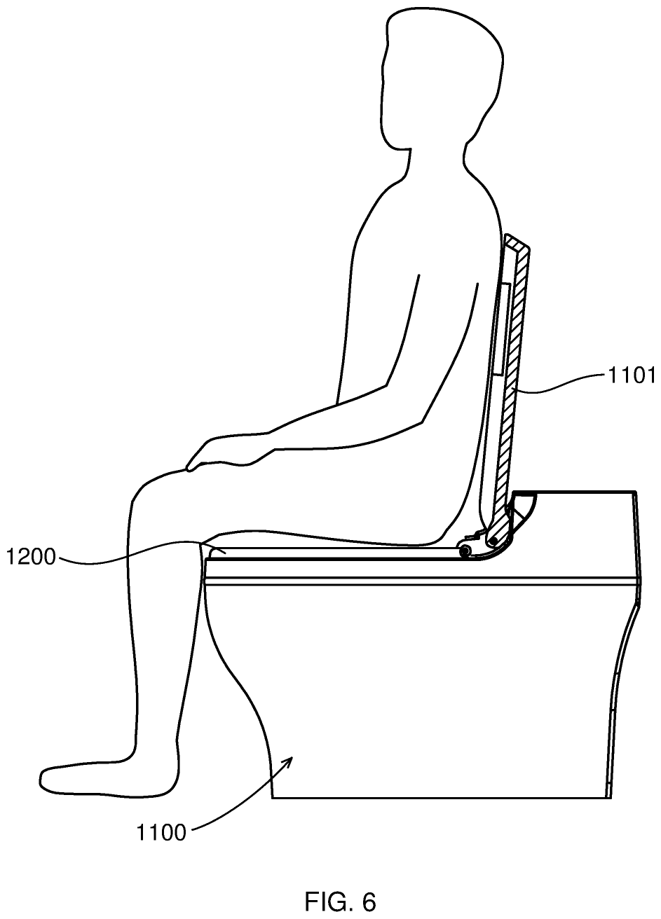

In various exemplary embodiments, the lid 1101 also includes one or more second health and wellness sensors 1102B. The second sensors 1102B may interact with the user's back while using the toilet, as shown in FIG. 6. Such second sensors 1102B may include a stethoscope, an ultrasound probe, an echocardiogram probe, a temperature sensor, a durometer, an electrocardiogram lead, and a ballistocardiography sensor. Physical contact or close proximity with the user is necessary for many of these sensors. In various exemplary embodiments, the lid may contain microphones to measure the excretion profile or other sounds.

In various exemplary embodiment, the lid 1101, sensors 1102B, or other components in contact with the user's skin may include an ultrasound sensor. Ultrasound is used to image internal organs and may be used to track the size and shape of internal organs in the torso of a user detecting, for example, if an organ becomes swollen (e.g., appendicitis, infections).

In various exemplary embodiment, the lid 1101, sensors 1102B, or other components in contact with the user's skin may include a durometer. This is used to test skin hardness which may be indicative of conditions such as scleroderma which may harm blood vessels, internal organs and the digestive tract in addition to the skin. In various exemplary embodiments, the lid may also contain imaging sensors that detect conditions such as scaly skin, discoloration, changes or growth of moles that may be symptoms of more serious conditions.

In various exemplary embodiments, the lid 1101 includes a seal 1104 that forms an airtight or watertight seal creating a chamber including the bowl 1300. In such embodiments, the toilet 1100 may include second sensors 1102A in the lid or other sensors in the toilet system for testing the air in the chamber and/or passages for drawing air out of the chamber for testing elsewhere in the system. Such sensors or tests may include spectrometers, chromatographs, FET, nano-FET, MOSFET, mass spectrometers, thermometers (including IR and thermocouples), rheometers, pH detectors, and scent detectors. Drawing air out of the chamber may also aid in drying the bowl between uses to reduce the risk of cross-sample contamination. The air passages may also be used to increase or decrease pressure in the airtight chamber. This may be advantageous in preparing excreta for sampling or other purposes. In other embodiments, the seal may be water-tight or splash-proof for bowl cleansing.

In various exemplary embodiments, the seat 1200 includes one or more second health and wellness sensors 1207. Such second sensors 1207 may include a stethoscope, an ultrasound probe, an echocardiogram probe, a temperature sensor, a durometer, an electrocardiogram lead, and a ballistocardiography sensor.

In various exemplary embodiments, the inner surface of the lid 1101 may include an ultraviolet light source to disinfect the interior of the bowl or seat.

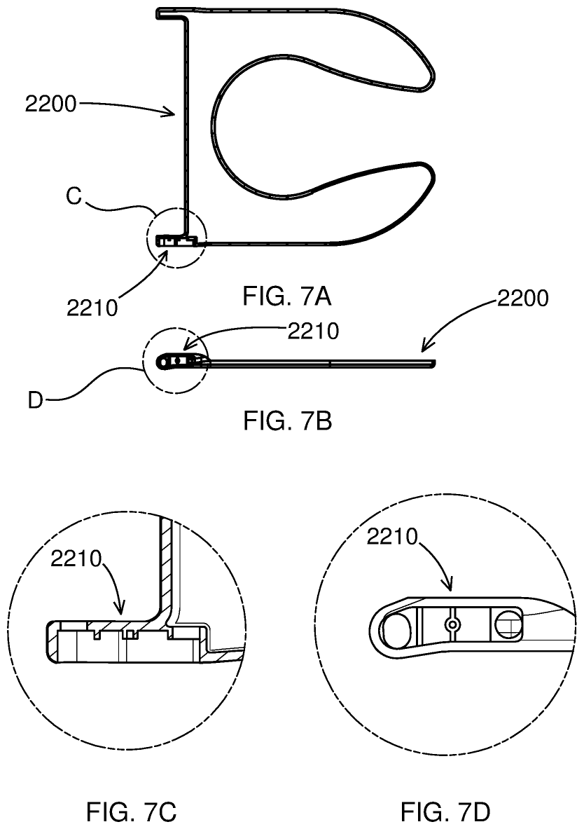

Referring to FIGS. 7A-D, another exemplary embodiment of a toilet seat 2200 with a floating hinge 2210 is shown. FIG. 7D shows the interior of hinge 2210. Some portions of the floating hinge 2210 may be integral to the seat or may be attached to the toilet seat 2200 with connectors (e.g., screws or bolts).

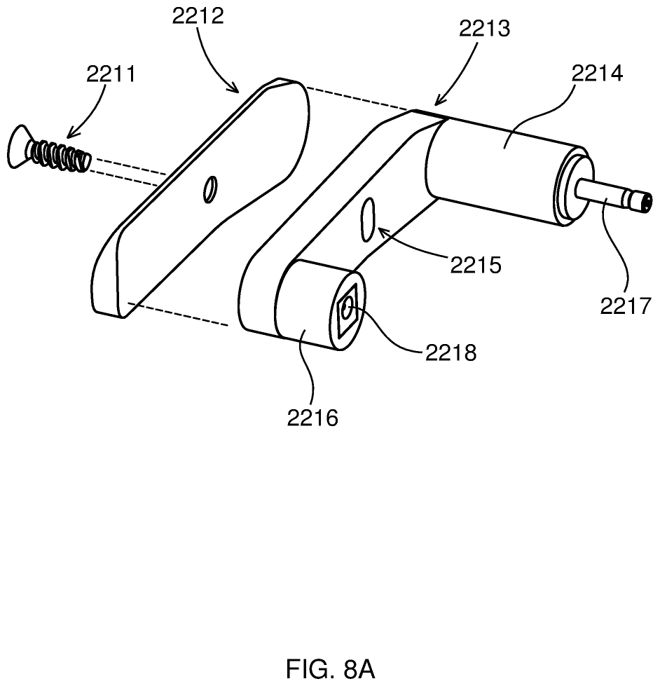

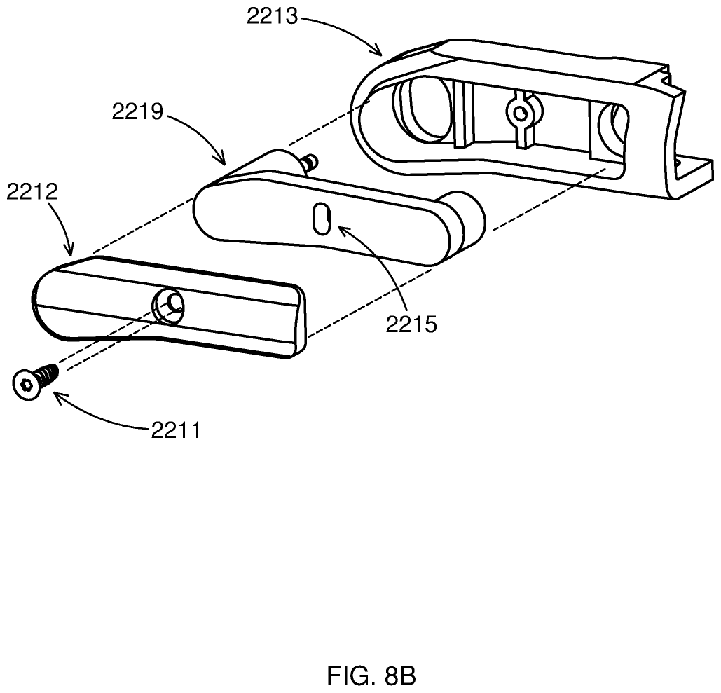

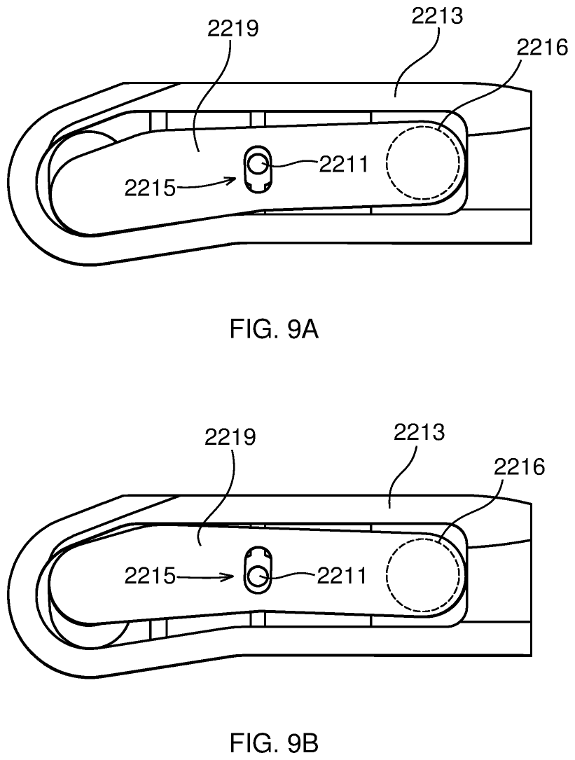

Referring to FIGS. 8A-B, show the components of an exemplary floating hinge 2210. FIG. 8A shows one side of the hinge 2210 assembly. FIG. 9B shows it from the opposite side. In various exemplary embodiments, the hinge 2210 comprises a floating hinge case 2213, pivot bar 2219, and floating hinge cover 2212 connected by screw 2211. Screw 2211 connects the cover 2212 to the case 2213 enclosing the pivot bar 2219. In some embodiments, the screw 2211 also connects the hinge 2210 to the seat 2200.

In various exemplary embodiments, pivoting bar 2219 comprises seat axle 2214, screw slot 2215, pivot axle 2216, and electrical connectors 2217 and 2218. Seat axle 2214 is adapted to connect to the toilet and provides a point around which the seat 2200 is raised and lowered. The pivot axle 2216 rotatably connects the pivot bar 2219 to the hinge case 2213. As will be described in more detail below, the screw slot 2215 allows the pivot bar 2219 to pivot around the pivot axle 2216 without interacting with screw 2211.

In various exemplary embodiments, connectors 2217 and 2218 are included to provide a supply of electrical power to components within the seat and lid, such as load cells, health and wellness sensors, processors, controllers, and wireless communication devices, among others. Connectors 2217 and 2218 may also provide data connections to similar components elsewhere in the system. In preferred embodiments, the electrical wiring connecting connectors 2217 and 2218 are molded into the pivot bar 2219.

Referring to FIGS. 9A-B, illustrate the range of motion through which the floating hinge 2210 is able to float. Pivot bar 2219 rotates around the axis of pivot axle 2216 within hinge casing 2213. Screw slot 2215 is adapted to allow this without interference from screw 2211. Seat axle 2214 is able to move up and down relative to the hinge casing 2213. When a user sits on the toilet seat, the weight of the user may cause the seat to lower slightly causing pivot axle 2216 to lower. Seat axle 2214 does not move because it is fixed relative to the toilet body. Because of the movement of pivot axle 2216, pivot bar 2219 moves relative to hinge casing 2213 such that seat axle 2214 moves upwardly and does not transfer the weight of the user to the toilet frame.

In various exemplary embodiments, the toilet seat lid comprises one or more health and wellness sensors that are adapted to interact with a seated user or to analyze the contents of the bowl with the lid closed. The sensors may interact with the user's back while using the toilet or may be collecting data with the lid closed. The interior of the bowl may be illuminated with visual, infrared, near infrared, ultraviolet, or other wavelengths by light sources.

In various exemplary embodiments, the toilet lid is powered through a connection to the toilet body. In a preferred embodiment, the connection may be made through the hinge between the lid and the body. In another embodiment, power is supplied through wires separate from the hinges. In various exemplary embodiments, the data connection to the lid may use the same wiring connections as the power or may be a wireless connection.

In various exemplary embodiments, urine and feces are separated into different collection basins for independent weighing and/or other analysis. In various exemplary embodiments, cameras estimate the volume of the fecal matter and use the estimated volume to estimate density.

In various exemplary embodiments, the toilet is capable of determining the total weight of excreta and the separate weights of solids and liquids. For example, weight may be measured after completion of an excreta event. Liquids may be allowed to exit the bowl and a new weight measurement taken of the remaining solids. The difference in weight provides the weight of liquid excreta. Alternatively, the urine may be drained off at a known flow rate or through a flow meter to measure urine volume leaving only solids to be weighed.

In various exemplary embodiments, a default position for the bowl and/or seat may be selected by the user. In such embodiments, the system will return to this position after being used by someone with a different preferred or optimal position. In various exemplary embodiments, the smart toilet may be programmed with separate default configurations for children and adults.

Preferably, the actuators are controlled by a digital controller that coordinates the actions of the actuators to thereby provide a smooth lifting and lower at the desired speed. More preferably, the controller is programmable, so that users can program the lifting and lowering of the seat to their preference. Even more preferably, the toilet includes a user identification module, so that the user is automatically identified as he or she approaches or sits on the toilet.

In accordance with the present disclosure, a smart toilet that includes mechanical, hydraulic, power, and data connections to accommodate a combination of health measuring tools is provided. An electrical connection to provide power to the health measuring tools is also provided.

In various exemplary embodiments, the smart toilet includes at least one sensor that analyzes imaging data. A processor analyzes the data to attempt to compare the user to known users for identification purposes. If a known user is identified, the position of the bowl and/or seat is automatically adjusted to the preferred position of the user. If the user is unknown or does not have a record preferred position, the processor analyzes the physical characteristics of the user (e.g., height, waist height, length of upper and lower legs) and adjusts the position of the bowl and/or seat accordingly. In some embodiments, facial recognition is used to identify users.

In various preferred embodiments, the system may identify a user based on their face, hand, or foot. In various preferred embodiments, the sensor may include a CCD (charge-coupled device) or MOS (metal oxide semiconductor), including CMOS (complementary metal oxide semiconductor). The sensor can be used, with proper calibration such as taking the data at a known distance, to measure the length of major bones. This data can then be used to customize the toiler position for an unknown user.

In various exemplary embodiments, the toilet system includes sensors for measuring the user's weight. This includes separate sensors for determining user weight on the seat and user weight on the user's feet. The user's weight that is supported by their feet is preferably measured using a platform adjacent to the toilet on which the user's feet are placed while sitting and while being lowered or raised from the toilet.

In the preferred embodiments, the toilet also includes health assessment devices supported by the frame. Examples of such devices include imaging cameras, flow spectrometers, volume measurement devices, body weight sensors, and gas analyzers. Toilets with such devices are described in the patents and published applications cited in the Background section above.

In various exemplary embodiments, the seat and bowl may be lifted together such that the toilet may be used as a urinal. In such a configuration, the system may be designed to flush with a reduced volume of water.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. An analytical toilet comprising:
a bowl for receiving excreta from a user;
a lid adapted to cover the bowl; and
a first health and wellness sensor in the lid for measuring a characteristic of the user's excreta.

2. The analytical toilet of claim 1 wherein the first health and wellness sensor comprises at least one of CCD, MOS, CMOS, spectrometer, chromatograph, microphone, thermometer, pH detector, and scent detector.

3. The analytical toilet of claim 1 wherein the characteristic of excreta is color, clarity, consistency, content, or temperature.

4. The analytical toilet of claim 1, further comprising a second health and wellness sensor in the lid for measuring a characteristic of the user.

5. The analytical toilet of claim 4 wherein the second health and wellness sensor comprises at least one of CCD, MOS, CMOS, electrode, microphone, load cell, pressure gauge, thermometer, and durometer.

6. The analytical toilet of claim 3 wherein the lid is adapted to contact the second health and wellness sensor with the user's torso.

7. The analytical toilet of claim 4 wherein the characteristic of the user is skin hardness, skin color, skin appearance, pulse, temperature, and the appearance or changes to moles.

8. The analytical toilet of claim 1 wherein the lid forms a seal between the bowl and the lid forming a sealed chamber.

9. The analytical toilet of claim 7 wherein the sealed chamber is air-tight.

10. The analytical toilet of claim 8 further comprising a gas exhaust to evacuate gas from the airtight chamber.

11. The analytical toilet of claim 10 further comprising an analytical tool for examining gas evacuated from the airtight chamber.

12. The analytical toilet of claim 11 wherein the analytical tool comprises one or more of chromatograph, spectrometry, mass spectrometry, thermometers, pH detectors, and scent detectors.

13. The analytical toilet of claim 1 further comprising at least one load cell supporting the bowl to measure the weight of the excreta.

14. The analytical toilet of claim 13 wherein data from the at least one weight sensor is used to measure the pressure of the closed lid on the bowl.

15. The analytical toilet of claim 1 further comprising a manifold system for transporting fluids within the toilet.

16. The analytical toilet of claim 15 wherein the fluids comprise at least one of excreta samples, gas from the airtight chamber, water, reagents, cleaners, disinfectants, air, high pressure water, and hot water.

17. The analytical toilet of claim 15 wherein the fluids are transported to at least one analytical tool.

18. The analytical toilet of claim 1 further comprising handles for the user to grasp.

19. The analytical toilet of claim 18 wherein the handles further comprise at least one health and wellness sensor for detecting health and wellness data from the user.

20. The analytical toilet of claim 19 wherein the at least one sensors comprise at least one of electrocardiograph, electrode, load cell, pressure gauge, PPG, thermometer, and durometer.
